(19) United States
(12) Patent Application Publication
(10) Pub. No.: US 2022/0387467 A1
(43) Pub. Date: Dec. 8, 2022

(54) USE OF SGLT2 INHIBITORS TO TREAT PRIMARY BILLIARY CHOLANGITIS

(71) Applicant: AVOLYNT, Research Triangle Park, NC (US)

(72) Inventors: William Owen WILKISON, Raleigh, NC (US); James Trinca GREEN, Raleigh, NC (US)

(21) Appl. No.: 17/776,033

(22) PCT Filed: Nov. 20, 2020
(86) PCT No.: PCT/US2020/061487

§ 371 Date: May 11, 2022

Related U.S. Application Data
(60) Provisional application No. 62/939,155, filed on Nov. 22, 2019.

(51) Int. Cl. A61K 31/7056; A61P 1/16; A61K 9/00

(52) U.S. Cl. CPC A61K 31/7056; A61P 1/16; A61K 9/0053

(57) ABSTRACT

Compositions of SGLT2 inhibitors and their use for treating primary biliary cholangitis (PBC) are described here. The SGLT2 inhibitor compositions, including oral dosage forms, contain a therapeutically effective dose of a SGLT2 inhibitor for preventing, partially ameliorating or fully ameliorating symptoms of PBC, including of the hepatic encephalopathy, development of varices, jaundice, variceal bleeding cholangiocarcinoma, hepatocellular carcinoma, evidence of cirrhosis, and colorectal cancer.

### CROSS REFERENCE TO RELATED APPLICATIONS

This application claims priority to U.S. Application No. 62/939,155, filed on Nov. 22, 2019, which is incorporated by reference herein in its entirety.

### FIELD OF THE INVENTION

The invention relates to compositions and methods associated with using an inhibitor of the sodium/glucose transporter 2 (“SGLT2”) to treat primary biliary cholangitis (“PBC”).

### BACKGROUND

Cholestasis is a condition in which the flow of bile from the liver to the duodenum is slowed or blocked. Cholestasis may be divided conveniently into two types: intrahepatic cholestasis, inside the liver, where bile formation is disturbed by conditions such as various diseases, extended intravenous nutrition, or as a side effect of certain drugs (such as some antibiotics); and extrahepatic cholestasis, occurring outside the liver, typically where the flow of bile is obstructed by a mechanical partial or complete closure of the bile duct, such as by bile duct tumors, cysts, bile duct stones, strictures, or pressure on the bile duct; though primary biliary cholangitis (PBC) may be intrahepatic or extrahepatic. Common symptoms of cholestasis include fatigue, pruritus (itching), jaundice, and xanthoma (deposits of cholesterol-rich material under the skin). The effects of cholestasis are profound and widespread, leading to worsening liver disease with systemic illness, liver failure, and the need for liver transplantation.

Intrahepatic cholestatic diseases include, in order of decreasing frequency, primary biliary cholangitis (PBC, formerly known as primary biliary cirrhosis), primary sclerosing cholangitis (PSC), progressive familial intrahepatic cholestasis (PFIC), and Alagille syndrome (AS),

PBC is an autoimmune disease of the liver marked by the slow progressive destruction of the small bile ducts of the liver, with the intralobular ducts affected early in the disease. When these ducts are damaged, bile builds up in the liver (cholestasis) and over time damages the tissue, which can lead to scarring, fibrosis and cirrhosis. Recent studies have shown that it may affect up to 1 in 3,000-4,000 people, with a sex ratio at least 9:1 female to male. There is no cure for PBC, and liver transplantation often becomes necessary; but medications such as ursodeoxycholic acid (UDCA, ursodiol) and Ocaliva (obeticholic acid, OCA) to reduce cholestasis and improve liver function, cholestyramine to absorb bile acids, modafinil for fatigue, and fat-soluble vitamins (vitamins A, D, E, and K, since reduced bile flow makes it difficult for these vitamins to be absorbed) may slow the progression to allow a normal lifespan and quality of life.

UDCA and Ocaliva are the only drugs approved in the United States to treat PBC. Japanese researchers have reported that the addition of bezafibrate, a peroxisome proliferator-activated receptor alpha. (PPAR alpha) and pregnane X receptor agonist, to UDCA is helpful in treating patients who are refractory to UDCA monotherapy, improving serum biliary enzymes, cholesterol (C), and triglycerides (TGs).

PSC is a chronic cholestatic liver disease characterized by intra- or extrahepatic biliary duct inflammation and fibrosis, eventually leading to cirrhosis. The underlying cause of the inflammation is believed to be autoimmunity; and about three-fourths of patients with PSC have inflammatory bowel disease, usually ulcerative cholitis, though this is reported to vary by country, as is the prevalence (generally reported at about 1 in 10,000) and sex ratio (generally reported as predominately male). Standard treatment includes UDCA, which has been shown to lower elevated liver enzyme numbers in people with PSC, but has not improved liver survival or overall survival; and also includes antipruritics, cholestyramine, fat-soluble vitamins, and antibiotics to treat infections (bacterial cholangitis). In a study reported in 2009, long-term high-dose UDCA therapy was associated with improvement in serum liver tests in PSC but did not improve survival and was associated with higher rates of serious adverse events. Liver transplantation is the only proven long-term treatment.

PFIC refers to a group of three types of autosomal recessive disorders of childhood associated with intrahepatic cholestasis: deficiency of familial intrahepatic cholestasis 1 (PFIC-1), deficiency of bile salt export pump (PFIC-2), and deficiency of multidrug resistance protein 3 (PFIC-3). They have a combined incidence of 1 in 50,000-100,000. The onset of the disease is usually before age 2, with PFIC-3 usually appearing earliest, but patients have been diagnosed with PFIC even into adolescence. Patients usually show cholestasis, jaundice, and failure to thrive; and intense pruritus is characteristic. Fat malabsorption and fat soluble vitamin deficiency may appear. Biochemical markers include a normal .gamma.-glutamyl transpeptidase (GGT) in PFIC-1 and PFIC-2, but a markedly elevated GGT in PFIC-3; while serum bile acid levels are greatly elevated; though serum cholesterol levels are typically not elevated, as is seen usually in cholestasis, because the disease is due to a transporter as opposed to an anatomical problem with biliary cells. The disease is typically progressive without liver transplantation, leading to liver failure and death in childhood; and hepatocellular carcinoma may develop in PFIC-2 at a very early age. Medication with UDCA is common; supplemented by fat-soluble vitamins, cholestyramine, and pancreatic enzymes in PFIC-1.

AS, also known as Alagille-Watson syndrome, syndromic bile duct paucity, and arteriohepatic dysplasia, is an autosomal dominant disorder associated with liver, heart, eye and skeletal abnormalities, as well as characteristic facial features; with an incidence of about 1 in 100,000. The liver abnormalities are narrowed and malformed bile ducts within the liver; and these result in obstruction of bile flow, causing cirrhosis (scarring) of the liver. AS is predominately caused by changes in the Jagged1 gene, located on chromosome 20. In 3-5% of cases, the entire gene is deleted (missing) from one copy of chromosome 20; in the remainder, there are changes or mutations in the Jagged1 DNA sequence. In a very small number of cases, less than 1 percent, changes in another gene, Notch2, result in AS. In about one-third of the cases, the mutation is inherited; in about two-thirds, the mutation is new in that case. There is no cure for AS, though the severity of liver disease typically peaks by 3 to 5 years of age and often resolves by 7 to 8 years of age. In some people, the hepatic disease will progress to end-stage liver disease and may require liver transplantation; approximately 15% of patients with AS require liver transplantation. A number of different medications, for example UDCA, have been used to improve bile flow and reduce itching, and many patients are given high doses of fat-soluble vitamins.

Alkaline phosphatase (ALP) and GGT are key markers of cholestasis. While an elevation of one of them alone does not indicate cholestasis, and other parameters would be needed for confirmation, elevation in both ALP and GGT is indicative of cholestasis; and a decrease in both indicates improvement of cholestasis. Thus ALP and GGT levels serve as biochemical markers for the presence of biliary pathophysiology present in intrahepatic cholestatic diseases, and ALP level has been used as a primary outcome marker in clinical studies of intrahepatic diseases such as PBC (including in the studies leading to FDA approval of obeticholic acid). With that goal in mind, novel approaches for treating PBC are described below. These developments are based on the unexpected observation that an SGLT2 inhibitor, remogliflozin etabonate, prevents the progression of PBC disease pathology.

### SUMMARY OF THE INVENTION

The invention relates to treating primary biliary cholangitis (PBC) with at least one SGLT2 inhibitor. Methods and compositions associated with the invention improve or maintain clinical outcomes in PBC-afflicted individuals following the administration of an SGLT2 inhibitor, including clinical symptoms such as ascites accumulation, hepatic encephalopathy, development of varices, jaundice, variceal bleeding, cholangiocarcinoma, hepatocellular carcinoma, evidence of cirrhosis, and colorectal cancer.

Abnormal liver function tests can be used to identify PBC patients that can benefit from SGLT2 inhibitor therapy. For example, PBC patients with blood plasma levels greater than the upper limit of normal (ULN) for one or more of Alkaline Phosphatase, Alanine aminotransaminase, γ-Glutamyl transpeptidase, Aspartate aminotransaminase, and total Bilirubin can can be treated with compositions and methods of the invention, as can PBC patients that present with one or more of liver fibrosis, inflammatory bowel disease, and abnormal liver stiffness.

SGLT2 inhibitors can be administered orally in either an immediate release (“IR”) or a delayed release (“DR”) dosage form, or in a biphasic dosage form containing an IR and DR phase.

### BRIEF DESCRIPTION OF THE FIGURES

FIG. 1A shows liver and biliary pathology in an H&E stained liver section harvested from a wild type mouse. Normal liver histochemistry is observed. PV=branch of the portal vein; HA=branch of the hepatic artery. BD=bile duct. Bar=100 μm.

FIG. 1B shows the presence of multiple portal tracts in an H&E stained liver section harvested from an untreated TIA mouse at 11 weeks. Inflammation is centered around bile ducts, and is accompanied with bile ductular proliferation (multiple bile duct profiles per portal tract; arrows). PV=branch of the portal vein. Bar=100 μm.

FIG. 1C shows the obliteration (oBD; arrowhead) of a portal tract by inflammation in an H&E stained liver section harvested from an untreated TIA mouse at 18 weeks. HA=branch of the hepatic artery. BD=bile duct. PV=branch of the portal vein. Bar=100 μm.

FIG. 1D shows activated immune cells in an H&E stained liver section harvested from an untreated TIA mouse at 18 weeks, that have surrounded, attacked and damaged bile duct epithelial cells (black arrowhead). Bar=100 μm.

FIG. 1E shows the development of onion skin fibrosis of bile ducts in a TIA mouse at 18 weeks of age. Bar=100 μm.

FIG. 2A shows hepatic parenchyma inflammation in an H&E stained liver section harvested from an untreated TIA mouse at 11 weeks. PV indicates portal vein. Bar=500 μm.

FIG. 2B shows biliary inflammation around bile ducts following in an H&E stained liver section harvested from an untreated TIA mouse at 11 weeks. PV indicates portal vein. Asterisks (*) indicate bile ducts. Bar=50 μm.

FIG. 2C shows inflammation at the interface between the hepatic parenchyma and the portal tracts in an H&E stained liver section harvested from an untreated TIA mouse at 11 weeks. PV indicates portal vein. Bar=50 μm.

FIG. 2D shows a decrease in periportal and biliary inflammation in an H&E stained liver section harvested from a TIA mouse at 11 weeks, that received 0.03% Remo in chow, beginning at 4 weeks of age. PV indicates portal vein. Bar=500 μm.

FIG. 2E shows a decrease in proliferation of bile ductules in an H&E stained liver section harvested from an untreated TIA mouse at 11 weeks that received 0.03% Remo in chow, beginning at 4 weeks of age. Asterisks (*) indicate bile ducts. PV indicates portal vein. Bar=50 μm.

FIG. 3 shows a plot of inflammation scores based on the histological examination of H&E stained liver sections harvested from TIA mice at 11 weeks that had been fed either standard chow, or a 0.03% remogliflozin-formulated standard chow, for 7 weeks. Scores were based on the degree of fibrosis, bile ductular proliferation or ductopenia, portal inflammation, lobular inflammation, interface hepatitis, presence of cholangitis, or periductal fibrosis/onion-skinning, as described in Table 1.

### DETAILED DESCRIPTION

Compositions and methods for using an SGLT2 inhibitor for treating individuals afflicted with primary biliary cholangitis (PBC) are described herein. Therefore, the invention relates to methods of administering an SGLT2 inhibitor to an individual, typically a human subject, or in other words, a patient, in an amount effective to treat PBC. SGLT2 inhibitors used in methods according to the invention generally, but not necessarily, belong to the gliflozin class of SGLT2 inhibitors. More particular examples of SGLT2 inhibitors that can be used in methods of the invention include, but are not limited to: Canagliflozin (sold under the tradenames, Invokana® and Sulisent®); Dapagliflozin (sold under the tradename, Farxiga®); Empagliflozin (sold under the tradename, Jardiance®); Ertugliflozin (sold under the tradename, Steglatro®); Ipragliflozin (sold under the tradename, Suglat®); Tofogliflozin (currently being developed by Chugai Pharma in collaboration with Kowa and Sanofi); Luseogliflozin (currently being developed by Taisho Pharmaceutical under the tradename Lusefi®); Remogliflozin (currently being developed by Avolynt, Inc. and sold under the tradename Remo® and Remozen®); Sotogloflozin (Also known as LX4211, and currently being developed by Lexicon Pharmaceuticals); Licogliflozin (also know as LIK-066, and currently being developed by Novartis), TFC-039 (currently being developed by Sirona Biochem); Sergliflozin; and salts of the foregoing SGLT2 inhibitors.

SGLT2 is a low affinity, high capacity sodium-glucose cotransporter located mainly in the S1 segment of the proximal tubule of the kidney. SGLT2 inhibition improves glucose clearance from the bloodstream, by increasing urinary glucose excretion. However, SGLT2 protein is also expressed in the central vein and biliary tract of the liver. Therefore, the administration of a SGLT2 inhibitor to a PBC patient can cause the inhibition of SGLT2 activity in liver of a PBC patient, which, in turn, halts the progession of PBC.

Typical PBC-related clinical outcomes include, for example, progression to cirrhosis, liver failure, death and liver transplantation. PBC-related clinical complications include, for example, ascites, hepatic encephalopathy, development of varices, jaundice, variceal bleeding, cholangiocarcinoma, hepatocellular carcinoma, evidence of cirrhosis, and colorectal cancer. A method for treating PBC with a SGLT2 inhibitor in a subject can improve clinical outcomes or clinical complications of PBC.

A patient suffering from PBC who can benefit from SGLT2 inhibitor therapy can have abnormal liver function tests. For example, the patient can have an abnormal ALP test. A PBC patient's blood serum ALP level may be greater than the upper limit of normal (ULN), for example, 1.5 times ULN, 1.6 times ULN, 2 times ULN, 2.5 times ULN, 3 times ULN, 4 times ULN, or a range of 1.5 to 10 times ULN or a range of 3 to 12 times ULN. Other abnormal liver function tests which can be exhibited by a patient suffering from PBC include a tests for blood levels or functions of ALT, GGT, AST, and total bilirubin.

A PBC patient that benefits from SGLT2 inhibitor therapy may also present with liver fibrosis or IBD, or both. Alternatively, a PBC patient undergoing SGLT2 inhibitor therapy may present with liver fibrosis or IBD, or both, but demonstrate normal liver function, based on liver function tests. The IBD can be: Ulcerative colitis (“UC”); Crohn's disease; or Indeterminate, undifferentiated or unclassified IBD (“IBDU”). A patient suffering from PBC who can benefit from SGLT2 inhibitor therapy can also have abnormal liver stiffness. Accordingly, a method according to the invention can be used for treating a PBC patient with a liver stiffness transient elastography (“TE”) score of ≤20 kPa, ≤18 kPa, ≤16 kPa, ≤15 kPa, ≤14 kPa, ≤13 kPa.

A therapeutically effective amount of an SGLT2 inhibitor according to a method of the invention may be an amount sufficient to reduce, delay or prevent progression of one or more PBC-related clinical complications, liver failure, or death. A therapeutically effective amount of an SGLT2 inhibitor can be administered in a single dose to a subject in need thereof, including a single dose that is administered as part of a treatment regimen that includes multiple adminstrations of the SGLT2 inhibitor. A therapeutically effective dose of an SGLT2 inhibitor according to a method of the invention may also be administered once daily, twice daily, three times daily, or more than three times daily, to a subject in need thereof.

A therapeutically effective amount of an SGLT2 inhibitor for treating PBC according to the invention may, for example, be determined based on various PBC disease metrics. Therefore, a therapeutically effective amount of a SGLT2 inhibitor may be a dosage amount sufficient to: Maintain, improve, or normalize a clinical disease assessment score; or Maintain, reduce, or normalize the level of a marker of liver function or pathology in the subject. Alternatively, a therapeutically effective amount of SGLT2 inhibitor administered to a subject can also be a dosage amount sufficient to: Maintain or improve an Ishak fibrosis staging score; Maintain, reduce, or normalize serum ALP; Maintain or improve an Ishak necroinflammatory grading score; Maintain, improve, or normalize an Amsterdam Cholestatic Complaints Score (“ACCS”); Maintain, improve, or normalize 5-D itch scale; Maintain, improve, or normalize the time to progression to cirrhosis, as assessed by a TE score; Maintain, improve, or normalize the time to PBC-related clinical outcomes or clinical complications; Maintain, improve, or normalize a subject's collagen proportional area (“CPA”); Maintain, improve, or normalize Enhanced Liver Fibrosis (“ELF”) score, as assessed by an algorithm using tests for serum concentrations of procollagen-III aminoterminal propeptide, tissue inhibitor of matrix metalloproteinase-1 and hyaluronic acid; Maintain, improve, or normalize a liver stiffness score, as assessed by TE or magnetic resonance elastography (“MRE”); or Maintain, improve, or normalize Mayo PBC risk score, or any combination thereof.

In general, a therapeutically effective amount of an SGLT2 inhibitor, also known as a dosage amount, is, but is not limited to, an amount of the SGLT2 inhibitor that is administered daily, and ranges from about 5 mg to about 2000 mg. A therapeutically effective dosage amount of remogliflozin etabonate according to the invention may be, for example, 50 mg, 100 mg, 200 mg 250 mg, 400 mg, 800 mg, 1000 mg, or 2000 mg, administered once or twice daily. A therapeutically effective dosage amount of empagliflozin according to the invention, by contrast, may be, for example, 5 mg, 10 mg, 15 mg 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, or 95 mg administered once or twice daily. Similarly, a therapeutically effective dosage amount of dapagliflozin according to the invention are may be, for example, 5 mg, 10 mg, 15 mg 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, or 95 mg administered once or twice daily, while a therapeutically effective dosage amount of canagliflozin according to the invention are may be, for example, 100 mg, 300 mg, or 600 mg administered once or twice daily.

As indicated above, a therapeutically effective dose of a SGLT2 inhibitor can be administered in a unit dose or multiple doses. The dosage can be determined by methods known in the art and can be dependent, for example, upon the individual's age, sensitivity, tolerance and overall well-being. A clinician or pharmacist of ordinary skill can determine appropriate dosing using the guidance provided herein and conventional methods. For example, the levels of a marker, such as, for example, ALP, in the individual being treated can be used as a metric to guide adjustments to a therapeutically effective dose of a SGLT2 inhibitor to achieve a desired reduction or normalization of the level of the marker.

Examples of modes of administration for a SGLT2 inhibitor include enteral routes, such as through a feeding tube or suppository, and parenteral routes, such as intravenous, intramuscular, subcutaneous, intraarterial, intraperitoneal, intravitreal administration, or orally. For example, a preferred mode of administration for the SGLT2 inhibitor, remogliflozin etabonate, is an oral route for the administration of oral dosage forms of remogliflozin etabonate.

Pharmaceutical compositions of the invention may be prepared by methods known in the pharmaceutical formulation art, for example, see Remington's Pharmaceutical Sciences, 22nd Ed., (Pharmaceutical Press, 2012), which is incorporated herein by reference. In a solid dosage form, a SGLT2 inhibitor may be admixed with at least one pharmaceutically acceptable excipient such as, for example: (a) sodium citrate; (b) dicalcium phosphate; (c) fillers or extenders, such as, for example, starches, lactose, sucrose, glucose, mannitol, and silicic acid; (d) binders, such as, for example, cellulose derivatives, starch, aliginates, gelatin, polyvinylpyrrolidone, sucrose, and gum acacia; (e) humectants, such as, for example, glycerol; (f) disintegrating agents, such as, for example, agar-agar, calcium carbonate, potato or tapioca starch, alginic acid, croscarmellose sodium, complex silicates, and sodium carbonate; (g) solution retarders, such as, for example, paraffin; (h) absorption accelerators, such as, for example, quaternary ammonium compounds; (i) wetting agents, such as, for example, cetyl alcohol, and glycerol monostearate, magnesium stearate; (j) adsorbents, such as, for example, kaolin and bentonite; and (k) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms, including oral dosage forms, may be prepared with coatings and shells, such as enteric coatings and others, as is known in the pharmaceutical art. They may contain pacifying agents, and can be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Liquid dosage forms may be aqueous, may contain a pharmaceutically acceptable solvent as well as traditional liquid dosage form excipients known in the art, which include, but are not limited to, buffering agents, flavorants, sweetening agents, preservatives, and stabilizing agents.

Oral dosage forms according to the invention are typically tablets or capsules. Tablets can be obtained by direct compression of the mixed components of a dosage form, including a therapeutically effective amount of an SGLT2 inhibitor, and selected excipients, like cellulose derivatives, metacrylates, chitosan, carboxymethylstarch, or mixtures thereof. For example, a compressed tablet according to the invention, can be prepared by granulating a SGLT2 inhibitor with microcrystalline cellulose and croscarmellose sodium with a water and povidone solution. The resulting granules are dried, milled, and then blended with mannitol, microcrystalline cellulose, and croscarmellose. The blend is lubricated with magnesium stearate and compressed. A compressed IR tablet according to the invention, which, for example, contains a dose of 350 mg of remogliflozin etabonate, can be orally administered to a subject to reach a maximum remogliflozin plasma concentration (Cₘₐₓ) of 160 ng/mL at 1 hr post-ingestion, and plasma clearance to 40 ng/mL after 3 hrs. Indeed, Tₘₐₓ for an IR remogliflozin etabonate oral dosage form according to the invention occurs at 1 hour, or less, following ingestion of the dosage form by a subject.

Alternatively, an oral dosage form according to the invention can be soft or hard capsule. For example, a capsule dosage form according to the invention may include SGLT2 inhibitor-layered pellets prepared by coating microcrystalline cellulose spheres with an aqueous suspension containing micronized SGLT2 inhibitor, povidone, and purified water. Capsules are typically manufactured from animal-derived gelatin or plant-derived hydroxypropyl methylcellulose (HPMC). The size of a capsule for an oral dosage form of the invention can be any size that is sufficient to contain a therapeutically effective dose of a SGLT2 inhibitor and excipient components. For example, the capsule can be a size 5, 4, 3, 2, 1, 0, 0E, 00, 000, 13, 12, 12e1, 11, 10, 7, or Su07. Capsules are filled using any suitable techniques.

In various methods according to the invention, an oral dosage form of a SGLT2 inhibitor according to the invention may be an immediate release (“IR”) formulation, or a dosage form designed to release the SGLT2 inhibitor after a period of delay, commonly known as a delayed release (“DR”), extended release, or modified release formulation. Alternatively, it may be appropriate for a SGLT2 inhibitor to partitioned into IR and DR components within a single dosage form.

An IR formulation or formulation component can include one or more hydrophilic materials, or one or more hydrophobic materials, or a combination of hydrophilic and hydrophobic materials. Hydrophilic and hydrophobic materials can be polymers. Examples of a hydrophilic polymers include, but are not limited to: hydroxypropylmethylcellulose, hydroxypropylcellulose, sodium carboxymethylcellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, and potassium pectinate. Examples of hydrophobic polymer that are available for inclusion in an oral dosage form according to the invention include, but are not limited to: Ethyl cellulose; Hydroxyethyl cellulose; An amino methacrylate copolymer; A methacrylic acid copolymer; A methacrylic acid acrylic acid ethyl ester copolymer; A methacrylic acid ester neutral copolymer; A dimethyl-amino-ethyl-methyl-methacrylate-methacrylic acid ester copolymer; A vinyl methyl ether or maleic anhydride copolymer; and Salts and esters thereof. Hydrophobic polymers may also be selected from: A wax, including bees wax, carnuba wax, microcrystalline wax and ozokerite; A fatty alcohol, including cetostearyl alcohol, stearyl alcohol, cetyl alcohol or myristyl alcohol; and A fatty acid ester, including glyceryl monostearate, glycerol monooleate, acetylated monoglycerides, tristearin, tripalmitin, cetyl esters wax, glyceryl palmitostearate, glyceryl behenate, and hydrogenated castor oil.

DR dosage forms can be tablets, filled capsules or layered pellets layered with SGLT2 inhibitor, which are coated with a DR coating, also known as an enteric coating. A DR coating protects an oral dosage form according to the invention from the harsh, acidic environment of the stomach, so that release of the therapeutically effective dose of a SGLT2 inhibitor is delayed until the dosage form reaches the small intestine. Any DR coatings of oral dosage forms of the invention are applied to a sufficient thickness such that the entire coating does not dissolve in the gastrointestinal fluids at pH below about 5. A DR coating typically includes a polymer, such as an aqueous dispersion of anionic polymers with methacrylic acid as a functional group like the product sold as Eudragit® L30D-55 (Evonik Industries). A DR coating can also optionally include a plasticizer, such as triethyl citrate, an anti-tacking agent, such as talc, and a diluent, such as water. For example, a coating composition used to coat and oral dosage form of the invention can contain about 42% (wt %) of an aqueous dispersion of anionic polymers with methacrylic acid as a functional group; about 1.25 wt % of a plasticizer; about 6.25 wt % of an anti-tacking agent; and about 51 wt % of a diluent. Another example of a coating composition for an oral dosage form of the invention, particularly when a large-scale preparation is preferred, an appropriate amount of an anionic copolymer based on methacrylic acid and ethyl acrylate, such as Eudragit® L100-55, is used in place of Eudragit® L30D-55. Conventional coating techniques such as spray or pan coating are employed to apply coatings. For example, a coating composition can be applied to capsules of the invention by using a Procept® coating machine and Caleva® mini coater air suspension coating machine to coat the capsules until they experience a 10% to 18% weight gain.

### Examples

The following Examples describe the utilization of a murine model of liver damage to assess the effectiveness of a treatment regimen based on the oral administration of remogliflozin etabonate. The murine model is based on mice that are deficient for the expression of tumor necrosis factor alpha (“TNFα”), interleukin 10 (“IL-10”), and activation-induced cytidine deaminase (“AICDA”). As the mice are deficient in TNF, IL-10, and AICDA, they are referred to, herein, as “TIA” mice. These liver outcomes in these animals also closely resemble that of PBC.

TIA mice can exhibit ulcerative colitis (“UC”)-like symptoms and pathology, as well as develop inflammation of the liver and biliary tract that, histologically, resembles PBC and PSC in humans. Moreover, as AICDA is required for immunoglobulin (“Ig”) class switching, TIA mice lack IgG and IgA, a phenotype analogous to humans with hyper-IgM syndrome. Therefore, with the combination of AICDA deficiency with the risk factors associated with TNFα and IL-10 deficiencies, TIA mice also develop liver and biliary inflammation reminiscent of PSC and PBC symptoms in humans. Accordingly, the TIA model is useful for investigating mechanisms that act early in PBC pathogenesis, as well as treatments that can prevent progression to PBC and PSC.

Example 1. Orally-administered remogliflozin etabonate reduces inflammatory cell infiltration, bile ductular proliferation, and interface hepatitis in TIA mice. TIA mice were created by first breeding TNFα knock out (“KO”) C57BL/6 mice, (strain B6.129S-Tnf^{tm1Gkl}/J, stock #005540, Jackson Laboratories, Bar Harbor, Me.) with IL-10 KO mice (strain B10.129P2(B6)-IL10^{tm1Cgn}/J, Stock No. 002251, Jackson Laboratories) to produce a population of mice that were deficient in TNFα and IL-10. Because mice with a TNFα −/− and IL10 −/− genotype spontaneously develop inflammatory bowel disease (“IBD”) (Hale 2012), a condition associated with poor breeding success (Nagy 2016), the mice needed for further breeding to generate an AICDA population, were generated by breeding offspring with a TNFα and and IL10+/− genotype with AICDA −/− mice, which were obtained from Dr. Tasuku Honjo (Muramatsu 2000)), to produce a population of TNFα −/−, IL10 −/−, and AICDA+/−(“TI-hetA”) males and females. In turn, TI-hetA pairs were bred to generate populations that were 25% TIA mice, and 50% non-colitis-susceptible TI-hetA littermates that could be used as control populations. All populations were exposed to the same environment from birth. The mice were housed in polycarbonate micro-isolator cages, in individually ventilated racks, under barrier conditions that excluded all known pathogens, including Helicobacter pylori and Norovirus. Mice had ad libitum access to water, and to a standard diet (PicoLab Mouse Diet 20/5058, LabDiet, St. Louis, Mo., USA).

At four (4) weeks of age, TIA (40) and TI-hetA (22) mice were randomized into experimental groups that received either a standard diet (20 TIA and 12 het), or a standard diet formulated 0.03% remogliflozin etabonate (20 TIA and 10 het) (Avolynt Inc., USA). The mice were maintained on this diet for seven (7) weeks. Body weights were obtained three (3) times, weekly, to assess the general health of mice, and to track the development of inflammatory bowel disease (IBD). Glycosuria in the experimental groups was assessed by applying freshly voided urine directly to the glucose test patch on an Accutest® URS-10 urinary reagent test strips (Jant Pharmaceutical Corp., Encino, Calif., USA). Mice were humanely euthanized before reaching the experimental endpoint, of eleven (11) weeks of age, if they lost >15% body weight, or developed rectal prolapse.

To characterize biliary lesions in TIA mice at the end of the 7 week treatment period, liver tissue was obtained from the remogliflozin-treated, and untreated groups, for histologic examination. The excised liver tissue was fixed in Carnoy's fixative solution, and processed into paraffin blocks. The paraffin blocks were sectioned, and stained with Hematoxylin and eosin (H&E) for pathologic analysis. The H&E-stained sections were scored by an American Board of Pathology-certified pathologist. The pathologist was blinded to mouse identity, and used an inflammation scoring system that was based on a modification of previously described scoring systems. Inflammation scores were based on the degree of fibrosis, bile ductular proliferation or ductopenia, portal inflammation, lobular inflammation, interface hepatitis, presence of cholangitis, or periductal fibrosis/onion-skinning. Table 1 summarizes the scoring system used to assess the tissues in this study.

TABLE 1

| Histologic Parameter | Score | Description |
|---|---|---|
| Histologic stage of | 1 | Normal to slight enlargement of portal tracts |
| fibrosis, | 2 | Portal expansion and periportal fibrosis |
| (Ludwig 1986) | 3 | Septal and/or bridging fibrosis |
| | 4 | Cirrhosis |
| Ductular proliferation | 0 | Absent |
| | 1 | Rare |
| | 2 | Present in 5-30% of portal areas |
| | 3 | Present in 30-90% of portal areas |
| | 4 | Expansion of >90% of portal areas with numerous duct profiles |
| Ductopenia | 0 | Absent |
| | 1 | Absence of interlobular and septal bile duct in >50% of portal areas |
| Degree of portal | 0 | Absent |
| inflammation | 1 | Mild (some or all portal areas) |
| | 2 | Moderate (some or all portal areas) |
| | 3 | Severe (all portal areas) |
| Intralobular | 0 | Absent |
| inflammation | 1 | Mild (≤2 foci per 10X field) |
| | 2 | Moderate (3-5 foci per 10X field) |
| | 3 | Severe (>5 foci per 10X field) |
| Hepatocellular | 0 | Absent |
| mitoses | 1 | Present |
| Interface hepatitis | 1 | Absent |
| (″piecemeal | 2 | Focal inflammation present around a minority of portal triads |
| necrosis″) | 3 | Mild to moderate inflammation present around most portal triads |
| | 4 | Moderate inflammation continuous around <50% of tracts |
| | 5 | Moderate to severe inflammation continuous around >50% of tracts |
| Cholangitis | 0 | Absent |
| (inflammation in | 1 | Present |
| bile duct lumen) | | |
| Onion-skinning | 0 | Absent |
| (fibrosis around bile | 1 | Present |
| duct) | | |

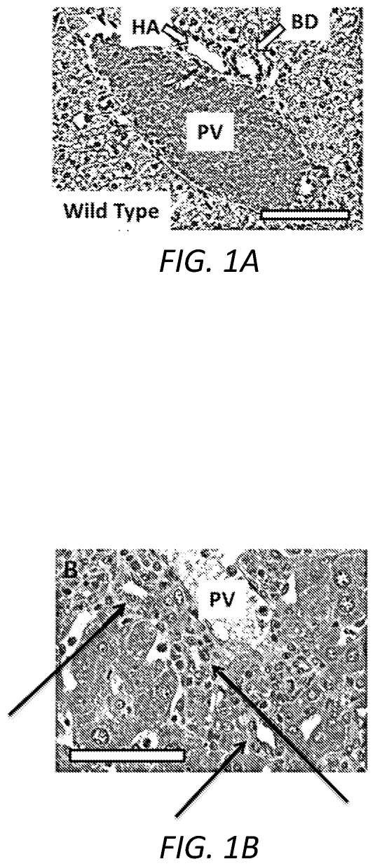

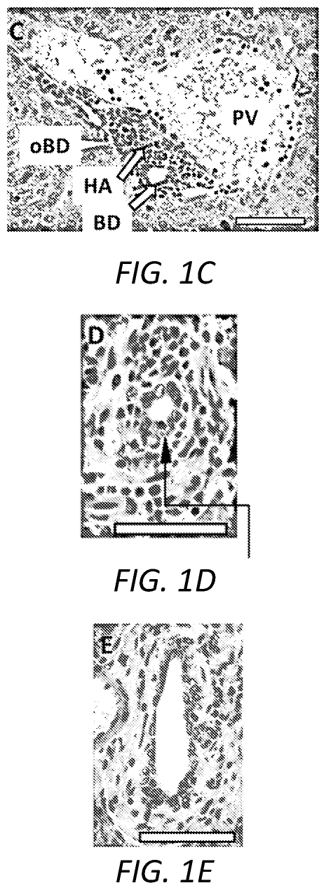

At 11 weeks, the livers of untreated TIA mice generally exhibited histologic lesions similar to those observed in PBC/PSC, including liver and biliary lesions, bile ductular proliferation, and interface hepatitis. See FIGS. 1A-B. Relatively few mice, however, formed major fibrotic lesions, such as onion skin fibrosis of bile ducts or ductopenia by 11 weeks, though such lesions were observed at 18 weeks (FIGS. 1C-E), and could be observed as early as 6 weeks in some TIA mice (data not shown). There were also relatively few mice that had developed cirrhosis, though macronodular cirrhosis was grossly observed in one TIA mouse at 28 weeks, before requiring euthanasia for weight loss (data not shown).

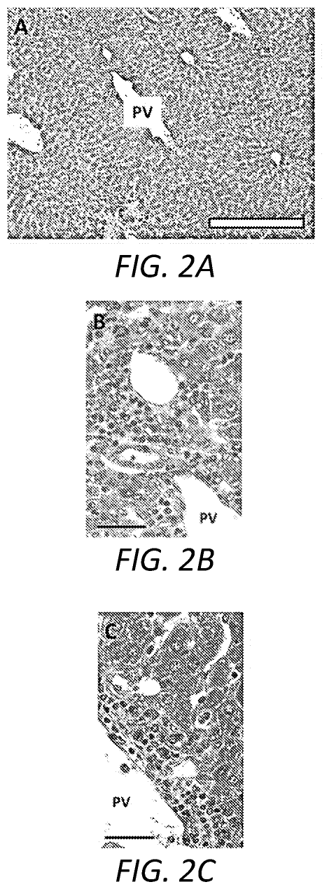

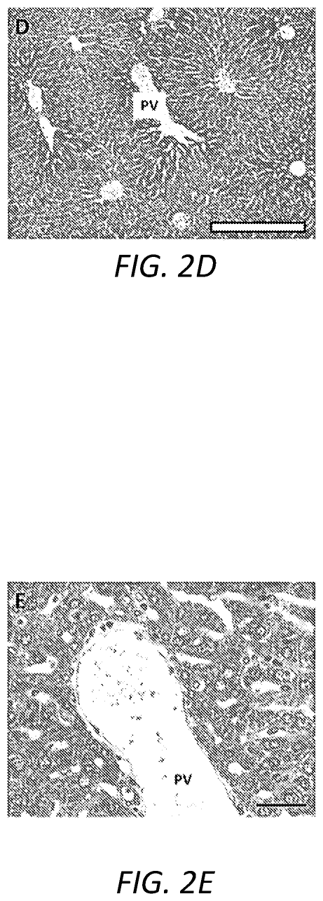

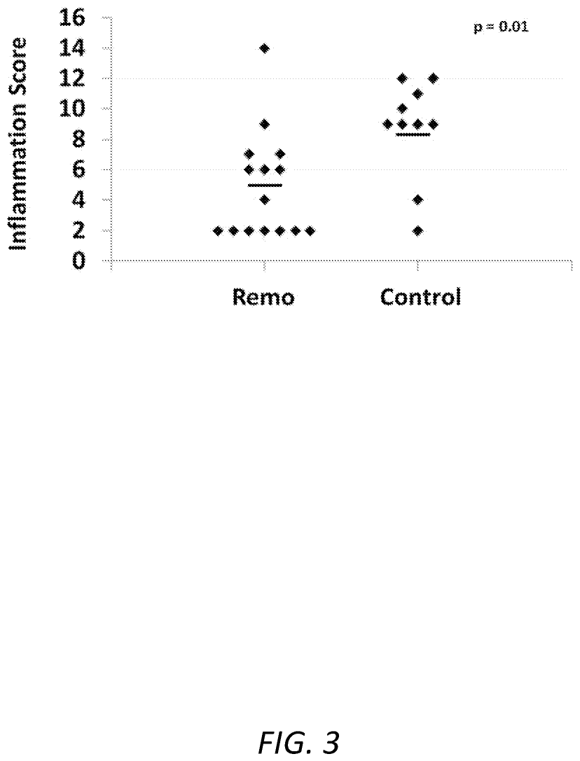

TIA mice, which fed on the remogliflozin etabonate-formulated diet for 7 weeks experienced markedly less development and progression of liver and biliary disease in comparison to TIA mice that remained on a standard diet. More specifically, the remogliflozin-fed TIA mice developed less inflammation at the interface between the hepatic parenchyma and the portal tracts (FIG. 2C), and periportal and biliary regions (FIG. 2D). The remogliflozin-fed TIA mice also experienced less proliferation of bile ductiles in comparison with untreated TIA mice. See FIG. 2E.

While there was no statistical difference in the number of TIA mice that required early euthanasia in the Remo group versus the control group in this study, survival curves of untreated TIA mice suggest a linear rate of death from 5-20 wks (n=90). Therefore, while not statistically significant, the trend toward decreased early death in the Remo group suggests that larger group sizes may uncover survival differences that this small study was not powered to detect.

Example 2. TIA mice demonstrate serologic evidence of of liver and/or biliary injury in TIA mice. A serum biochemical profile of TIA mice at 11 weeks was performed. Blood was drawn from euthanized animals into lithium heparin tubes, and a panel of analytes, including total protein, albumin, serum alkaline phosphatase (AP), alanine aminotransferase (ALT), and total bilirubin were measured using a Heska Dry Chem 7000 analyzer. Serum aspartate aminotransferase (AST) was measured in a separate test. In 50% of the mice, elevated levels of AP, ALT, and AST, which were at least 1.5× the upper limit of normal—levels considered to be indicative of cholestasis/liver damage, were detected. Histological analysis at 11 weeks, as described in Example 1, revealed considerable biliary and hepatic inflammation is present, but relatively little fibrosis. These serum biochemistry data are also suggestive of autoimmune hepatitis.

What is claimed is:

1. A method for treating primary biliary cholangitis (PBC), comprising administering an SGLT2 inhibitor, or a salt thereof.

2. The method according to claim 1, wherein the SGLT2 inhibitor, or a salt thereof, is administered orally.

3. The method according to claim 2, wherein the SGLT2 inhibitor, or salt thereof, is formulated as an oral dosage form.

4. The method according to claim 3, wherein the oral dosage form comprises:
a) SGLT2 inhibitor, or salt thereof,
b) at least one hydrophilic or hydrophobic material, or both, and
c) at least one pharmaceutically acceptable excipient.

5. The method according to claim 4, wherein the at least one hydrophilic or hydrophobic material is a polymer.

6. The method according to claim 3, wherein the oral dosage form is a tablet or a capsule.

7. The method according to claim 3, wherein the SGLT2 inhibitor, or a salt thereof, is present in an amount from 1 mg to 2000 mg.

8. The method according to claim 4, wherein the at least one hydrophilic or hydrophobic polymer is a hydrophilic polymer selected from the group consisting of hydroxypropyl methylcellulose, hydroxypropyl cellulose, sodium carboxymethyl cellulose, carboxymethyl cellulose calcium, ammonium alginate, sodium alginate, potassium alginate, calcium alginate, propylene glycol alginate, alginic acid, polyvinyl alcohol, povidone, carbomer, potassium pectate, and potassium pectinate.

9. The method according to claim 4, wherein the at least one hydrophilic or hydrophobic polymer is a hydrophobic polymer selected from the group consisting of ethyl cellulose, hydroxyethyl cellulose, amino methacrylate copolymer, methacrylic acid copolymers, methacrylic acid acrylic acid ethyl ester copolymer, methacrylic acid ester neutral copolymer, dimethylaminoethylmethyl methacrylate-methacrylic acid ester copolymer, vinyl methyl ether/maleic anhydride copolymer, and salts and esters thereof.

10. The method according to claim 4, wherein the at least one hydrophilic or hydrophobic polymer is a hydrophobic polymer selected from the group consisting of a wax, a fatty alcohol, and a fatty acid ester.

11. The method according to claim 10, wherein:
A. the wax is bees wax, carnauba wax, microcrystalline wax or ozokerite;
B. the fatty alcohol is cetostearyl alcohol, stearyl alcohol, cetyl alcohol or myristyl alcohol; and
C. the fatty acid ester is glyceryl monostearate, glycerol monooleate, acetylated monoglycerides, tristearin, tripalmitin, cetyl esters wax, glyceryl palmitostearate, glyceryl behenate or hydrogenated castor oil

12. The method according to claim 4, wherein the at least one pharmaceutically acceptable excipient is a binder, a filler, a lubricant, a preservative, a stabilizer, an anti-adherent, a glidant, or a combination thereof.

13. The method according to claim 4, comprising the excipients: Povidone; Microcrystalline cellulose; Croscarmellose cellulose; and Magnesium stearate.

14. The method according to claim 3, wherein the oral dosage form is an enterically-coated tablet.
